(19) United States
(12) Patent Application Publication
(10) Pub. No.: US 2009/0192134 A1
(43) Pub. Date: Jul. 30, 2009

(54) Compounds

(76) Inventors: Premji Meghani, Loughborough (GB); Jeffrey Stonehouse, Loughborough (GB)

Correspondence Address: MORGAN LEWIS & BOCKIUS LLP, 1111 PENNSYLVANIA AVENUE NW, WASHINGTON, DC, 20004, US

(21) Appl. No.: 10/585,325

(22) PCT Filed: Jan. 19, 2005
(86) PCT No.: PCT/GB05/00180

§ 371 Date: Jul. 6, 2006

(30) Foreign Application Priority Data
Jan. 21, 2004 (GB) 0401269.6

(51) Int. Cl. A61K 31/53; C07D 251/52; A61P 35/00; C07D 403/12; C07D 413/12; A61K 31/5377

(52) U.S. Cl. 514/210.2; 544/208; 514/245; 544/209; 544/113; 514/236.2

(57) ABSTRACT

A compound of formula (1), or a pharmaceutically acceptable salt, solvate or in vivo hydrolysable ester thereof: and pharmaceutical compositions comprising these, all for use in the treatment of chemokine mediated diseases and disorders.

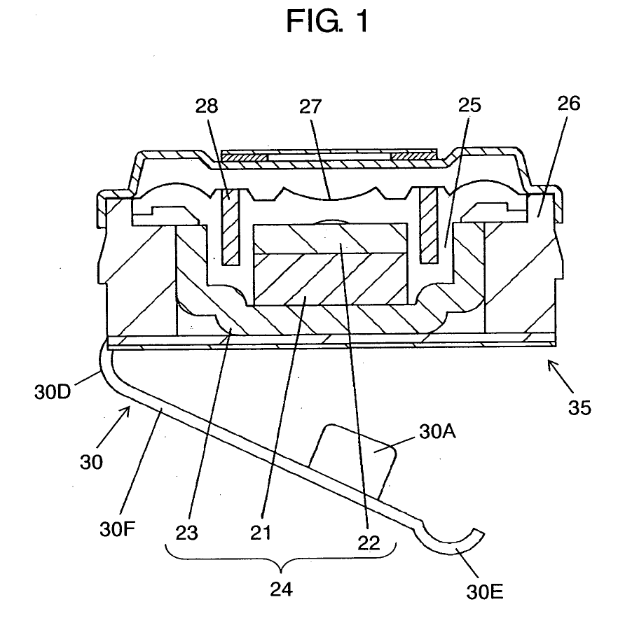

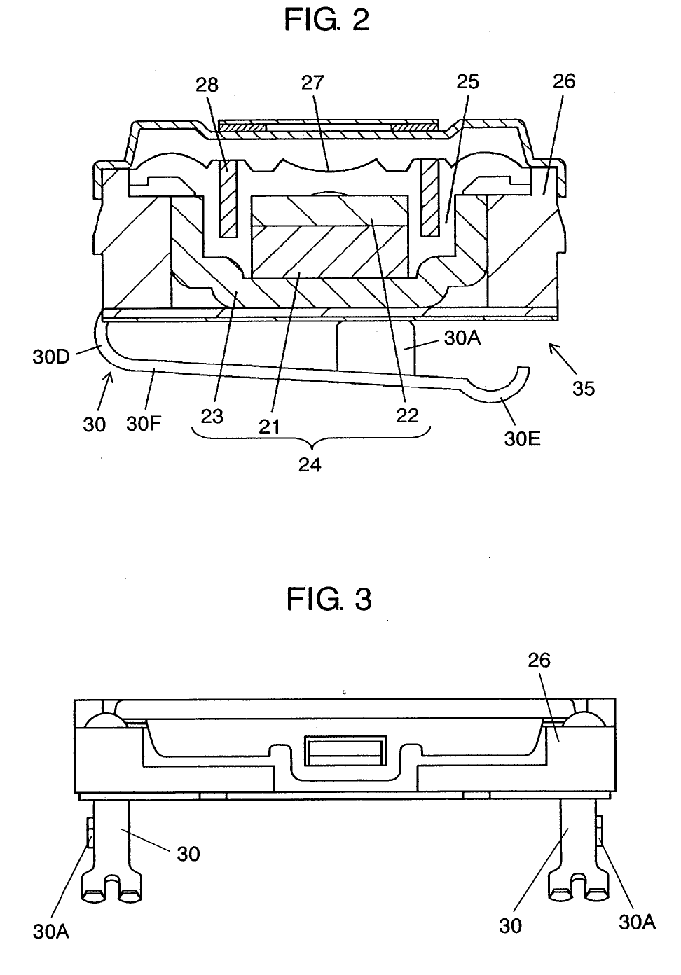

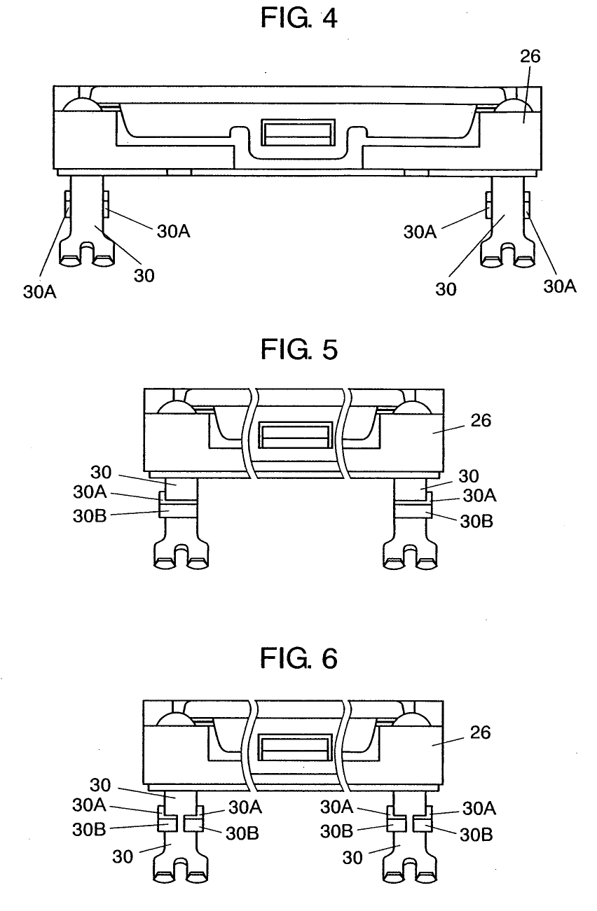

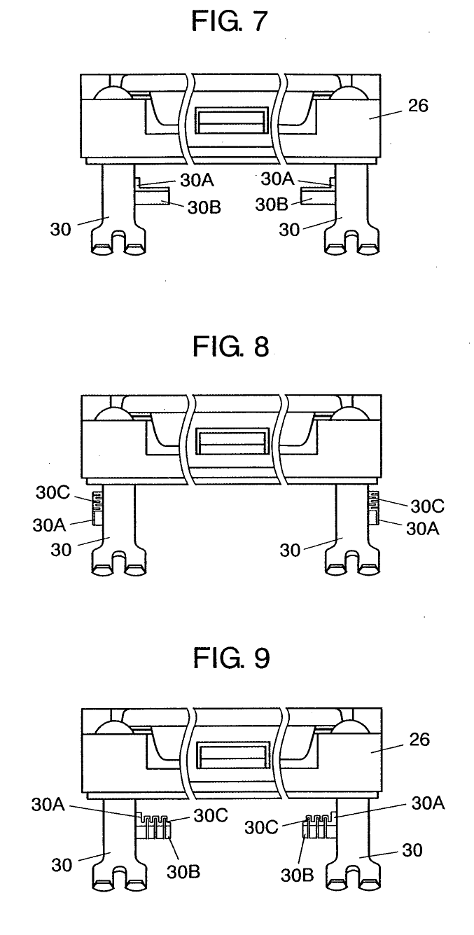

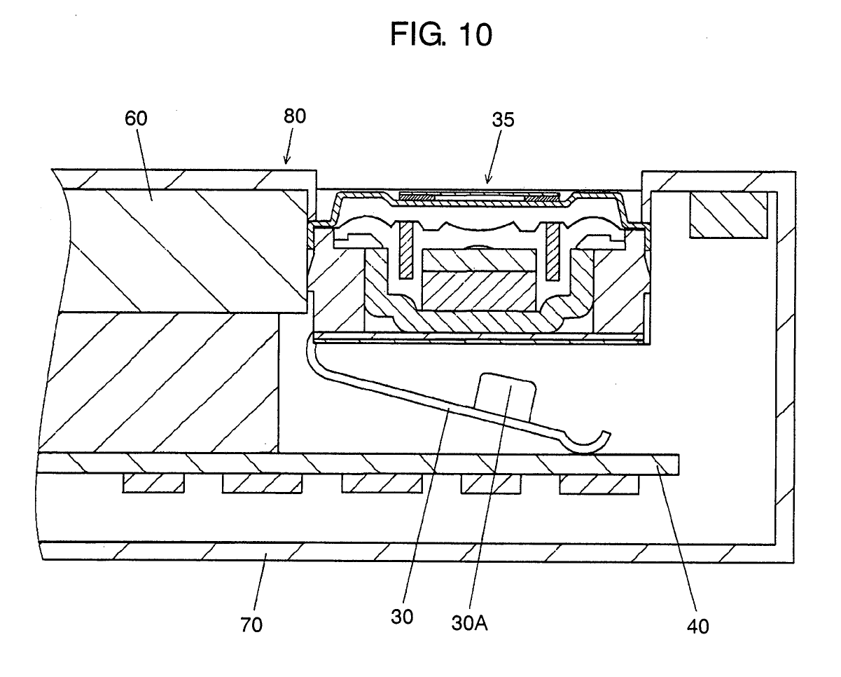

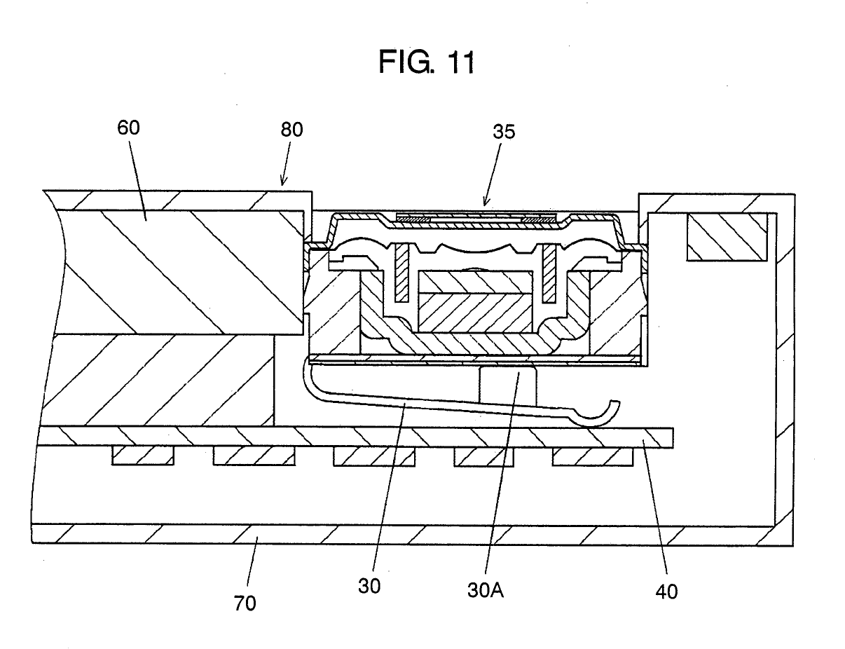

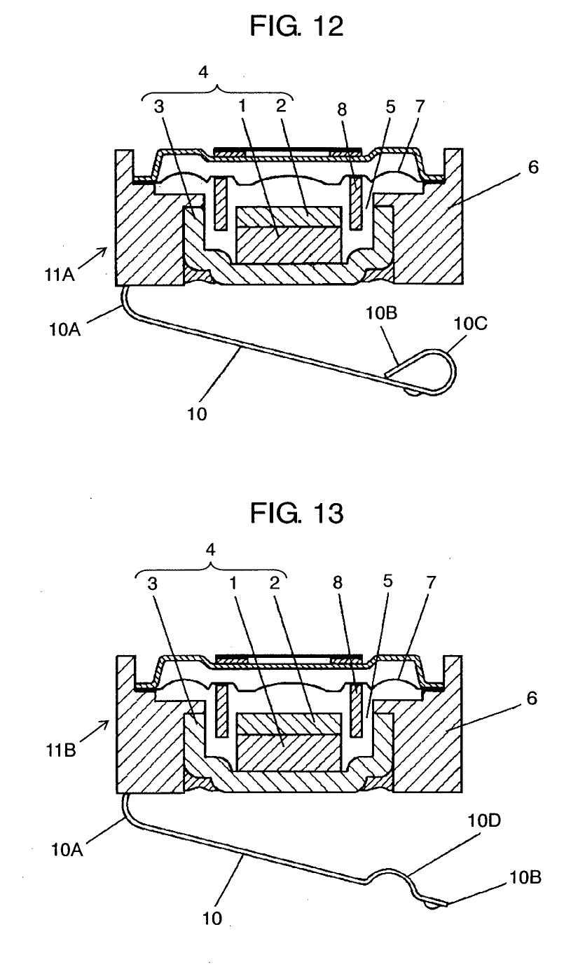

The present invention relates to certain heterocyclic compounds, processes and intermediates used in their preparation, pharmaceutical compositions containing them and their use in therapy.

Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved cysteine motif. At the present time, the chemokine superfamily comprises four groups exhibiting characteristic structural motifs, the C-X-C, C-C and C-X₃-C and XC families. The C-X-C and C-C families have sequence similarity and are distinguished from one another on the basis of a single amino acid insertion between the NH-proximal pair of cysteine residues. The C-X₃-C family is distinguished from the other two families on the basis of having a triple amino acid insertion between the NH-proximal pair of cysteine residues. In contrast, members of the XC family lack one of the first two cysteine residues.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils. Examples include human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

The C-X₃-C chemokine (also known as fractalkine) is a potent chemoattractant and activator of microglia in the central nervous system (CNS) as well as of monocytes, T cells, NK cells and mast cells.

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family. These receptors represent good targets for drug development since agents which modulate these receptors would be useful in the treatment of disorders and diseases such as those mentioned above.

The present invention provides compounds of formula (1), a pharmaceutically acceptable salt, solvate or in vivo hydrolysable ester thereof:

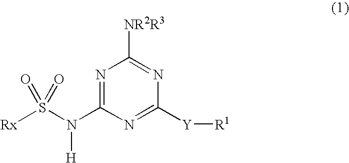

wherein Y is selected from a bond, —S—, —O—, —NR⁵—, —CF₂—CH₂—, —CF₂CF₂—, —CONR⁵—, phenyl or heteroaryl;
wherein R¹ is a group selected from C₃₋₇carbocyclyl, C₁₋₈alkyl, C₂₋₆alkenyl and C₂₋₆alkynyl; wherein the group is optionally substituted by 1, 2 or 3 substituents independently selected from fluoro, nitrile, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, phenyl or heteroaryl; wherein phenyl and heteroaryl are optionally substituted by 1, 2 or 3 substituents independently selected from halo, cyano, nitro, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, C₁₋₆alkyl and trifluoromethyl;
wherein R² is C₃₋₇carbocyclyl, optionally substituted by 1, 2 or 3 substituents independently selected from fluoro, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹;
or R² is a 3-8 membered ring optionally containing 1, 2 or 3 atoms selected from O, S, —NR⁸ and whereby the ring is optionally substituted by C₁₋₃alkyl or fluoro;
or R² is a phenyl or heteroaryl, each of which is optionally substituted by 1, 2 or 3 substituents independently selected from halo, cyano, nitro, —OR⁴, —NR⁵R⁶, —CONR⁵R⁶, —NR⁸COR⁹, —SO₂NR⁵R⁶, —NR⁸SO₂R⁹, C₁₋₆alkyl and trifluoromethyl;
or R² is a group selected from C₁₋₈alkyl, C₂₋₆alkenyl or C₂₋₆alkynyl wherein the group is substituted by 1, 2 or 3 substituents independently selected from hydroxy, amino, C₁₋₆alkoxy, C₁₋₆alkylamino, di(C₁₋₆alkyl)amino, N—(C₁₋₆alkyl)-N-(phenyl)amino, N—C₁₋₆alkylcarbamoyl, N,N-di(C₁₋₆alkyl)carbamoyl, N—(C₁₋₆alkyl)-N-(phenyl)carbamoyl, carboxy, phenoxycarbonyl, —NR⁸COR⁹, —SO₂R¹⁰, —SO₂NR⁵R⁶ and —NR⁸SO₂R⁹;
wherein R³ is hydrogen or independently R²;
R⁴ is hydrogen or a group selected from C₁₋₆alkyl and phenyl, wherein the group is optionally substituted by 1 or 2 substituents independently selected from halo, phenyl, —OR¹¹ and —NR¹²R¹³;
R⁵ and R⁶ are independently hydrogen or a group selected from C₁₋₆alkyl and phenyl wherein the group is optionally substituted by 1, 2 or 3 substituents independently selected from halo, phenyl, —OR¹⁴, —NR¹⁵R¹⁶, —COOR¹⁴, —CONR¹⁵R¹⁶, —NR¹⁵COR¹⁶, —SO₂R¹⁰, —SONR¹⁵R¹⁶ and NR¹⁵SO₂R¹⁶
or
R⁵ and R⁶ together with the nitrogen atom to which they are attached form a 4- to 7-membered saturated heterocyclic ring system optionally containing a further heteroatom selected from oxygen and nitrogen atoms, which ring is optionally substituted by 1, 2 or 3 substituents independently selected from phenyl, —OR¹⁴, —COOR¹⁴, —NR¹⁵R¹⁶, —CONR¹⁵R¹⁶, —NR¹⁵COR¹⁶, —SO₂R¹⁰, —SONR¹⁵R¹⁶, NR¹⁵SO₂R¹⁶ or C₁₋₆alkyl (optionally substituted by 1 or 2 substituents independently selected from halo, —NR¹⁵R¹⁶ and —OR¹⁷ groups);
R¹⁰ is hydrogen or a group selected from C₁₋₆alkyl or phenyl, wherein the group is optionally substituted by 1, 2 or 3 substituents independently selected from halo, phenyl, —OR¹⁷ and —NR¹⁵R¹⁶; and each of R⁷, R⁸, R⁹, R¹¹, R¹², R¹³, R¹⁴, R¹⁵, R¹⁶, R¹⁷ is independently hydrogen, C₁₋₆alkyl or phenyl;
R^{x} is trifluoromethyl, —NR⁵R⁶, phenyl, napthyl, monocyclic or bicyclic heteroaryl wherein a heteroring may be partially or fully saturated and one or more ring carbon atoms may form a carbonyl group, and wherein each phenyl or heteroaryl group is optionally substituted by 1, 2 or 3 substituents independently selected from halo, cyano, nitro, —OR⁴, —NR⁵R⁶, —CONR⁵R⁶, —COR⁷, —COOR⁷, —NR⁸COR⁹, —SR¹⁰, —SO₂R¹⁰, —SO₂NR⁵R⁶, —NR⁸SO₂R⁹, C₁₋₆alkyl or trifluoromethyl;
or R^{x} is a group selected from C₃₋₇carbocyclyl, C₁₋₈alkyl, C₂₋₆alkenyl and C₂₋₆alkynyl whereby the group is optionally substituted by 1, 2 or 3 substituents independently selected from halo, —OR⁴, —NR⁵R⁶, —CONR⁵R⁶, —COR⁷, —COOR⁷, —NR⁸COR⁹, —SR¹⁰, —SO₂R¹⁰, —SO₂NR⁵R⁶, —NR⁸SO₂R⁹, phenyl or heteroaryl; and wherein each phenyl or heteroaryl group is optionally substituted by 1, 2 or 3 substituents independently selected from halo, cyano, nitro, —OR⁴, —NR⁵R⁶, —CONR⁵R⁶, —COR⁷, —COOR⁷, —NR⁸COR⁹, —SR¹⁰, —SO₂R¹⁰, —SO₂NR⁵R⁶, —NR⁸SO₂R⁹, C₁₋₆alkyl or trifluoromethyl.

In particular Y is a bond; Y is —S—; Y is —O—; Y is —NR⁵; Y is —CF₂—CH₂—; Y is —CF₂CF₂—; Y is —CONR⁵—; Y is phenyl; or Y is heteroaryl.

Conveniently R¹ is benzyl or —CH₂CH₂OPh, or CH₂CH₂Ph where in each case the phenyl ring is optionally substituted by 1, 2 or 3 substituents independently selected from fluoro, chloro, bromo, methoxy, methyl and trifluoromethyl.

Conveniently R² is C₁₋₈alkyl optionally substituted by 1 or 2 hydroxy substituents and R³ is hydrogen.

Conveniently R^{x} is methyl, trifluoromethyl, 1-methylimidazolyl, 1,2-dimethylimidazolyl, N,N-dimethylamino, azetidinyl, pyrrolidinyl, morpholinyl or piperidinyl.

Certain compounds of formula (1) are capable of existing in stereoisomeric forms. It will be understood that the invention encompasses all geometric and optical isomers of the compounds of formula (1) and mixtures thereof including racemates.

The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Within the present invention it is to be understood that a compound of formula (1) or a salt, solvate or in vivo hydrolysable ester thereof may exhibit the phenomenon of tautomerism and that the formulae drawings within this specification can represent only one of the possible tautomeric forms. It is to be understood that the invention encompasses any tautomeric form and mixtures thereof and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It is also to be understood that certain compounds of formula (1) and salts thereof can exist in solvated as well as unsolvated forms such as, for example, hydrated forms. It is to be understood that the invention encompasses all such solvated forms.

The present invention relates to the compounds of formula (1) as hereinbefore defined as well as to the salts thereof. Salts for use in pharmaceutical compositions will be pharmaceutically acceptable salts, but other salts may be useful in the production of the compounds of formula (1) and their pharmaceutically acceptable salts. Pharmaceutically acceptable salts of the invention may, for example, include acid addition salts of the compounds of formula (1) as hereinbefore defined which are sufficiently basic to form such salts. Such acid addition salts include for example salts with inorganic or organic acids affording pharmaceutically acceptable anions such as with hydrogen halides (especially hydrochloric or hydrobromic acid of which hydrochloric acid is particularly preferred) or with sulphuric or phosphoric acid, or with trifluoroacetic, citric or maleic acid. Suitable salts include hydrochlorides, hydrobromides, phosphates, sulphates, hydrogen sulphates, alkylsulphonates, arylsulphonates, acetates, benzoates, citrates, maleates, fumarates, succinates, lactates, tartrates, oxalates, methanesulphonates or p-toluenesulphonates. Pharmaceutically acceptable salts of the invention may also include basic addition salts of the compounds of formula (1) as hereinbefore defined which are sufficiently acidic to form such salts. Such salts may be formed with an inorganic or organic base which affords a pharmaceutically acceptable cation. Such salts with inorganic or organic bases include for example an alkali metal salt, such as a lithium, sodium or potassium salt, an alkaline earth metal salt such as a calcium or magnesium salt, an ammonium salt or an organic amine salt, for example a salt with methylamine, dimethylamine, trimethylamine, triethylamine, piperidine, morpholine or tris-(2-hydroxyethyl)amine. Other basic addition salts include aluminium, zinc, benzathine, chloroprocaine, choline, diethanolamine, ethanolamine, ethyldiamine, meglumine, tromethamine or procaine.

The present invention further relates to an in vivo hydrolysable ester of a compound of formula (1). An in vivo hydrolysable ester of a compound of formula (1) which contains carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

Suitable pharmaceutically-acceptable esters for hydroxy include inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include C₁₋₁₀alkanoyl, for example acetyl; benzoyl; phenylacetyl; substituted benzoyl and phenylacetyl, C₁₋₁₀alkoxycarbonyl (to give alkyl carbonate esters), for example ethoxycarbonyl; di-(C₁₋₄)alkylcarbamoyl and N-(di-(C₁₋₄)alkylaminoethyl)-N—(C₁₋₄)alkylcarbamoyl (to give carbamates); di-(C₁₋₄)alkylaminoacetyl and carboxyacetyl. Examples of ring substituents on phenylacetyl and benzoyl include aminomethyl, (C₁₋₄)alkylaminomethyl and di-((C₁₋₄)alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O(C₁₋₆)alkyl—CO—, wherein R^{A} is for example, benzyloxy-(C₁₋₄)alkyl, or phenyl). Suitable substituents on a phenyl group in such esters include, for example, 4-(C₁₋₄)piperazino-(C₁₋₄)alkyl, piperazino-(C₁₋₄)alkyl and morpholino-(C₁₋₄)alkyl.

In this specification the term “alkyl” includes both straight-chain and branched-chain alkyl groups. However references to individual alkyl groups such as “propyl” are specific for the straight chain version only and references to individual branched-chain alkyl groups such as t-butyl are specific for the branched chain version only. For example, “C₁₋₃alkyl” includes methyl, ethyl, propyl and isopropyl and examples of “C₁₋₆alkyl” include the examples of “C₁₋₃alkyl” and additionally t-butyl, pentyl, 2,3-dimethylpropyl, 3-methylbutyl and hexyl. Examples of “C₁₋₈alkyl” include the examples of “C₁₋₆alkyl” and additionally heptyl, 2,3-dimethylpentyl, 1-propylbutyl and octyl. An analogous convention applies to other terms, for example “C₂₋₆alkenyl” includes vinyl, allyl, 1-propenyl, 2-butenyl, 3-butenyl, 3-methylbut-1-enyl, 1-pentenyl and 4-hexenyl and examples of “C₂₋₆alkynyl” includes ethynyl, 1-propynyl, 3-butynyl, 2-pentynyl and 1-methylpent-2-ynyl.

“C₃₋₇carbocyclyl” is a saturated, partially saturated or unsaturated, monocyclic ring containing 3 to 7 carbon ring atoms wherein a —CH₂— group can optionally be replaced by a —C(O)—. Suitable examples of “carbocyclyl” are cyclopropyl, cyclopentyl, cyclobutyl, cyclohexyl, cyclohexenyl, 4-oxocyclohex-1-yl and 3-oxocyclohept-5-en-1-yl.

The term “halo” refers to fluoro, chloro, bromo and iodo.

Examples of “C₁₋₆alkoxy” include methoxy, ethoxy, propoxy, isopropoxy, butyloxy, pentyloxy, 1-ethylpropoxy and hexyloxy. Examples of “C₁₋₆alkylamino” include methylamino, ethylamino, propylamino, butylamino and 2-methylpropylmino. Examples of “di(C₁₋₆alkyl)amino” include dimethylamino, N-methyl-N-ethylamino, diethylamino, N-propyl-N-3-methylbutylamino. Examples of “N—(C₁₋₆alkyl)-N-(phenyl)amino” include N-methyl-N-phenylamino, N-propyl-N-phenylamino and N-(2-methylbutyl)-N-phenylamino. Examples of “N—(C₁₋₆alkyl)carbamoyl” are N-methylcarbamoyl, N-ethylcarbamoyl and N-(2-ethylbutylcarbamoyl. Examples of “N—(C₁₋₆alkyl)-N-(phenyl) carbamoyl” include N-methyl-N-phenylcarbamoyl, N-butyl-N-phenylcarbamoyl and N-(3-methylpentyl)-N-(phenyl)carbamoyl. Examples of “N,N-di(C₁₋₆alkyl)carbamoyl” include N,N-dimethylcarbamoyl, N-methyl-N-ethylcarbamoyl and N-propyl-N-(2-methylbutyl)carbamoyl. Examples of “C₁₋₆alkylthio” include methylthio, ethylthio, propylthio, butylthio and 2-methylbutylthio.

“Heteroaryl” is a monocyclic or bicyclic aryl ring containing 5 to 10 ring atoms of which 1, 2, 3 or 4 ring atoms are chosen from nitrogen, sulphur or oxygen. Examples of heteroaryl include pyrrolyl, furanyl, thienyl, thiazolyl, isothiazolyl, oxazolyl, isoxazolyl, imidazolyl, pyrazolyl, triazolyl, tetrazolyl, pyridyl, pyrimidinyl, pyrazinyl, pyridazinyl, triazinyl, benzfuranyl, benzthieno, indolyl, benzimidazolyl, benzoxazolyl, benzthiazolyl, indazolyl, benzisoxazolyl, benzisothiazolyl, benztriazolyl, quinolinyl, isoquinolinyl and naphthiridinyl. Conveniently heteroaryl is selected from imidazolyl, pyrazolyl, thiazolyl, isoxazolyl, furanyl, thienyl, isoxazolyl, or indazolyl.

Examples of “a 3-8 membered ring optionally containing 1, 2 or 3 atoms selected from O, S and NR⁸” include oxetanyl, azetidinyl, benzodiazolyl, pyrrolidinyl, tetrahydrofuranyl, tetrahydrothiophenyl, tetrahydropyranyl, piperidinyl, piperazinyl, morpholinyl, homopiperidinyl and homopiperazinyl tetrahydrodioxanyl, such as oxetanyl, azetidinyl, pyrrolidinyl, tetrahydrofuranyl, tetrahydropyranyl, piperidinyl, piperazinyl, morpholinyl, homopiperidinyl and homopiperazinyl, further such as pyrrolidinyl, tetrahydropyridinyl, piperidinyl, piperazinyl, and morpholinyl.

Examples of “a 4- to 7-membered saturated heterocyclic ring system” include azetidinyl, pyrrolidinyl, piperidinyl, piperazinyl, homopiperazinyl and morpholinyl.

Where optional substituents are chosen from “1, 2 or 3” groups it is to be understood that this definition includes all substituents being chosen from one of the specified groups or the substituents being chosen from two or more of the specified groups. An analogous convention applies to substituents chosen from “1 or 2” groups.

Particular compounds of the invention include N-[4-[[(2,3-difluorophenyl)methyl]thio]-6-[[(1R)-2-hydroxy-1-methylethyl]amino]-1,3,5-triazin-2-yl]-methanesulfonamide; N-[4-[[(2,3-difluorophenyl)methyl]thio]-6-[[(1R)-2-hydroxy-1-methylethyl]amino]-1,3,5-triazin-2-yl]-1-azetidinesulfonamide N-[4-[[(2,3-difluorophenyl)methyl]thio]-6-[[(1R)-2-hydroxy-1-methylethyl]amino]-1,3,5-triazin-2-yl]-methanesulfonamide N-[4-[[(2,3-difluorophenyl)methyl]thio]-6-[[(1R)-2-hydroxy-1-methylethyl]amino]-1,3,5-triazin-2-yl]-1-azetidinesulfonamide 4-morpholinesulfonamide, N-[4-[[(2,3-difluorophenyl)methyl]thio]-6-[[(1R)-2-hydroxy-1-methylethyl]amino]-1,3,5-triazin-2-yl]-methanesulfonamide, N-[4-[[2-(2,3-difluorophenoxy)ethyl]thio]-6-[[(1R)-2-hydroxy-1-methylethyl]amino]-1,3,5-triazin-2-yl]-methanesulfonamide, 1,1,1-trifluoro-N-[4-[[(1R)-2-hydroxy-1-methylethyl]amino]-6-(2-phenylethyl)-1,3,5-triazin-2-yl]- and pharmaceutically acceptable salts, solvates or in vivo hydrolysable esters thereof.

Each of the above mentioned compounds and the pharmaceutically acceptable salt, solvate or in vivo hydrolysable ester thereof, individually is a particular aspect of the invention.

The present invention further provides a process for the preparation of compounds of formula (1) as defined above which comprises:

(a) treating a compound of formula (2):

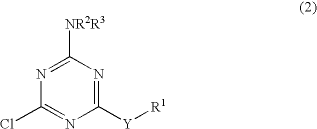

wherein Y, R¹, R² and R³ are as defined in formula (1) with sulfonamides (R^{x}SO₂NH₂) where R^{x} is as defined in formula (1).
and optionally thereafter (i), (ii), (iii), (iv), or (v) in any order:
i) removing any protecting groups;
ii) converting the compound of formula (1) into a further compound of formula (1)
iii) forming a salt
iv) forming a prodrug
v) forming an in vivo hydrolysable ester.

Reaction of compounds of formula (2) wherein Y, R¹, R² and R³ are as defined in formula (1) with sulfonamides (R^{x}SO₂NH₂) can be carried out in the presence of a suitable base, solvent and catalyst. Examples of suitable bases include metal carbonates such as those from ceasium, potassium, lithium or sodium. Most preferably ceasium carbonate is used. Suitable solvents include ethers such as tetrahydrofuran, 1,4-dioxane, glyme and diglyme. Preferably 1,4-dioxane is used. The temperature of the reaction can be performed between 10° C. and 120° C., preferably at 100° C. Examples of suitable catalysts include a suitable paladium(0) source such as palladium tris(dibenzylideneacetone)dipalladium(0) (Pd₂(dba)₃), or tetrakistriphenylphosphine (Pd(Ph₃)₄) (either in 0.01-0.5 mol equivalents) in the presence of a suitable ligand such as (9,9-dimethyl-9H-xanthene-4,5-diyl)bis[diphenyl-phosphine (Xantphos), or 2-dicyclohexyl-phosphino-2′-(N,N-dimethylamino)biphenyl (either in 0.01-0.5 mol equivalents). Preferably the catalyst combination is tris(dibenzylideneacetone)dipalladium(0) (Pd₂(dba)₃) with 4,5-bis(diphenylphosphino)-9,9-dimethylxanthene (Xantphos) in 0.01-0.5 mol equivalents in 1,4-dioxane at 100° C. with ceasium carbonate as the base.

Compounds of formula (2) wherein Y, R¹, R² and R³ are as defined in formula (1), can be prepared from compounds of formula (3) wherein R¹ are as defined in formula (1) and L is halogen by treatment with nucleophilic amines NR²R³ as defined in formula (1) in the presence of a suitable base and solvent.

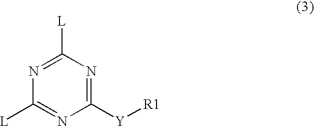

Examples of suitable bases include trialkylamines, such as triethylamine or N,N-diisopropylethylamine. Suitable solvents include ethers such as tetrahydrofuran, 1,4-dioxane, glyme and diglyme. The temperature of the reaction can be performed between 0° C. and 50° C. Preferably tetrahydrofuran is used at ambient temperature.

Compounds of formula (3) wherein Y is —S— and R¹ is as defined in formula (1) and L is halogen may be prepared by treating cyanuric chloride with a thiol of formula R¹SH wherein R¹ is defined as in formula (1) in presence of a suitable base and solvent. Suitable solvent include ethers such as tetrahydrofuran, 1,4-dioxane, glyme and diglyme. Suitable bases include trialkylamines, such as triethylamine or N,N-diisopropylethylamine. Preferably N,N-diisopropylethylamine is used as a base and tetrahydrofuran as the solvent is used at ambient temperature.

Compounds of formula (3) wherein Y is a bond and R¹ is as defined in formula (1) and L is halogen may be prepared by treating cyanuric chloride with a suitable Grignard reagent for example of formula R¹(CH₂)ₙMgL wherein L is a halogen and R¹ is defined as in formula (1) in presence of a suitable solvent such tetrahydrofuran or 1,4-dioxane. Preferably tetrahydrofuran at ambient temperature is used.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (1) may involve, at an appropriate stage, the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991).

A compound of formula (1) may be prepared from another compound of formula (1) by chemical modification. Examples of chemical modifications include standard alkylation, arylation, heteroarylation, acylation, sulphonylation, phosphorylation, aromatic halogenation and coupling reactions. These reactions may be used to add new substituents or to modify existing substituents. Alternatively, existing substituents in compounds of formula (1) may be modified by, for example, oxidation, reduction, elimination, hydrolysis or other cleavage reactions to yield other compounds of formula (1).

Novel intermediate compounds form a further aspect of the invention.

The compounds of formula (1) above may be converted to a pharmaceutically acceptable salt, solvate or in vivo hydrolysable ester thereof, as discussed above. The salt is preferably a basic addition salt.

The compounds of formula (1) have activity as pharmaceuticals, in particular as modulators of chemokine receptor activity, such as for example CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family; and especially as modulators of CXCR2 activity. Use of the compounds as modulators of each above mentioned receptor represents a separate and independent aspect of the invention.

The compounds of formula (1) may be used in the treatment (therapeutic or prophylactic) of conditions/diseases in human and non-human animals which are exacerbated or caused by excessive or unregulated production of chemokines. Examples of such conditions/diseases include (each taken independently):
(1) the respiratory tract—obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;
(2) bone and joints—rheumatoid arthritis, seronegative spondyloarthropathies (including ankylosing spondylitis, psoriatic arthritis and Reiter's disease), Behchet's disease, Sjogren's syndrome and systemic sclerosis;
(3) skin—psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis;
(4) gastrointestinal tract—Coeliac disease, proctitis, eosinopilic gastro-enteritis, mastocytosis, Crohn's disease, ulcerative colitis, indeterminate colitis, microscopic colitis, inflammatory bowel disease, irritable bowel syndrome, non-inflammatory diarrhea, food-related allergies which have effects remote from the gut, e.g., migraine, rhinitis and eczema;
(5) central and peripheral nervous system—Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia; polyneuropathies, e.g. Guillain-Barrè syndrome, chronic inflammatory demyelinating polyradiculoneuropathy, multifocal motor neuropathy, plexopathies; CNS demyelination, e.g. multiple sclerosis, acute disseminated/haemorrhagic encephalomyelitis, and subacute sclerosing panencephalitis; neuromuscular disorders, e.g. myasthenia gravis and Lambert-Eaton syndrome; spinal disorders, e.g. tropical spastic paraparesis, and stiff-man syndrome: paraneoplastic syndromes, e.g. cerebellar degeneration and encephalomyelitis; CNS trauma; migraine; and stroke.
(6) other tissues and systemic disease—atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura; post-operative adhesions, and sepsis.
(7) allograft rejection—acute and chronic following, for example, transplantation of kidney, heart, liver, lung, bone marrow, skin and cornea; and chronic graft versus host disease;
(8) cancers—especially non-small cell lung cancer (NSCLC), malignant melanoma, prostate cancer and squamous sarcoma, and tumour metastasis, non melanoma skin cancer and chemoprevention metastases;
(9) diseases—in which angiogenesis is associated with raised CXCR2 chemokine levels (e.g. NSCLC, diabetic retinopathy);
(10) cystic fibrosis;
(11) burn wounds & chronic skin ulcers;
(12) reproductive diseases—for example disorders of ovulation, menstruation and implantation, pre-term labour, endometriosis;
(13) re-perfusion injury—in the heart, brain, peripheral limbs and other organs, inhibition of atherosclerosis.

Thus, the present invention provides a compound of formula (1), or a pharmaceutically-acceptable salt, solvate or an in vivo hydrolysable ester thereof, as hereinbefore defined for use in therapy.

Preferably the compounds of the invention are used to treat diseases in which the chemokine receptor belongs to the CXC chemokine receptor subfamily, more preferably the target chemokine receptor is the CXCR2 receptor.

Particular conditions which can be treated with the compounds of the invention are cancer, diseases in which angiogenesis is associated with raised CXCR2 chemokine levels, and inflammatory diseases such as asthma, allergic rhinitis, COPD, rheumatoid arthritis, psoriasis, inflammatory bowel diseases, osteoarthritis or osteoporosis.

As a further aspect of the present invention, certain compounds of formula (1) may have utility as antagonists of the CX3CR1 receptor. Such compounds are expected to be particularly useful in the treatment of disorders within the central and peripheral nervous system and other conditions characterized by an activation of microglia and/or infiltration of leukocytes (e.g. stroke/ischemia and head trauma).

In a further aspect, the present invention provides a compound of formula (1), or a pharmaceutically acceptable salt, solvate or in vivo hydrolysable ester thereof, as hereinbefore defined for use as a medicament.

In a still further aspect, the present invention provides the use of a compound of formula (1), or a pharmaceutically acceptable salt, solvate or in vivo hydrolysable ester thereof, as hereinbefore defined for use as a medicament for the treatment of human diseases or conditions in which modulation of chemokine receptor activity is beneficial.

In a still further aspect, the present invention provides the use of a compound of formula (1), or a pharmaceutically acceptable salt, solvate or in vivo hydrolysable ester thereof, as hereinbefore defined for use as a medicament for the treatment of asthma, allergic rhinitis, cancer, COPD, rheumatoid arthritis, psoriasis, inflammatory bowel diseases, osteoarthritis or osteoporosis.

In a further aspect, the present invention provides the use of a compound of formula (1), or a pharmaceutically acceptable salt, solvate or in vivo hydrolysable ester thereof, as hereinbefore defined in the manufacture of a medicament for use in therapy.

In a still further aspect, the present invention provides the use of a compound of formula (1), or a pharmaceutically acceptable salt, solvate or in vivo hydrolysable ester thereof, as hereinbefore defined in the manufacture of a medicament for the treatment of human diseases or conditions in which modulation of chemokine receptor activity is beneficial.

In a still further aspect, the present invention provides the use of a compound of formula (1), or a pharmaceutically acceptable salt, solvate or in vivo hydrolysable ester thereof, as hereinbefore defined in the manufacture of a medicament for the treatment of asthma, allergic rhinitis, cancer, COPD, rheumatoid arthritis, psoriasis, inflammatory bowel diseases, osteoarthritis or osteoporosis.

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CXCR2) receptor, which comprises administering to a patient a therapeutically effective amount of a compound of formula, or a pharmaceutically acceptable salt, solvate or in vivo hydrolysable ester, as hereinbefore defined.

The invention also provides a method of treating an inflammatory disease, especially asthma, allergic rhinitis, COPD, rheumatoid arthritis, psoriasis, inflammatory bowel diseases, osteoarthritis or osteoporosis, in a patient suffering from, or at risk of, said disease, which comprises administering to the patient a therapeutically effective amount of a compound of formula (1), or a pharmaceutically acceptable salt, solvate or in vivo hydrolysable ester thereof, as hereinbefore defined.

For the above-mentioned therapeutic uses the dosage administered will, of course, vary with the compound employed, the mode of administration, the treatment desired and the disorder indicated.

The compounds of formula (1) and pharmaceutically acceptable salts, solvates or in vivo hydrolysable esters thereof may be used on their own but will generally be administered in the form of a pharmaceutical composition in which formula (1) compound/salt/solvate/ester (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (1), or a pharmaceutically acceptable salt, solvate or in vivo hydrolysable ester thereof, as hereinbefore defined, in association with a pharmaceutically acceptable adjuvant, diluent or carrier.

The invention further provides a process for the preparation of a pharmaceutical composition of the invention which comprises mixing a compound of formula (1), or a pharmaceutically acceptable salt, solvate or in vivo hydrolysable ester thereof, as hereinbefore defined, with a pharmaceutically acceptable adjuvant, diluent or carrier. The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compounds of the invention are administered orally.

In addition to their use as therapeutic medicines, the compounds of formula (1) and their pharmaceutically acceptable salts, solvate or in vivo hydrolysable esters are also useful as pharmacological tools in the development and standardisation of in vitro and in vivo test systems for the evaluation of the effect of chemokine modulation activity in laboratory animals such as cats, dogs, rabbits, monkeys, rats and mice, as part of the search for new therapeutic agents.

The invention further relates to combination therapies wherein a compound of formula (1) or a pharmaceutically acceptable salts, solvate or in vivo hydrolysable ester thereof, or a pharmaceutical composition or formulation comprising a compound of formula (1) is administered concurrently or sequentially with therapy and/or an agent for the treatment of any one of asthma, allergic rhinitis, cancer, COPD, rheumatoid arthritis, psoriasis, inflammatory bowel disease, irritable bowel syndrome, osteoarthritis or osteoporosis.

In particular, for the treatment of the inflammatory diseases rheumatoid arthritis, psoriasis, inflammatory bowel disease, irritable bowel syndrome, COPD, asthma and allergic rhinitis the compounds of the invention may be combined with agents such as TNF-α inhibitors such as anti-TNF monoclonal antibodies (such as Remicade, CDP-870 and D.sub2.E.sub7.) and TNF receptor immunoglobulin molecules (such as Enbrel.reg.), non-selective COX-1/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin), COX-2 inhibitors (such as meloxicam, celecoxib, rofecoxib, valdecoxib and etoricoxib) low dose methotrexate, lefunomide; ciclesonide; hydroxychloroquine, d-penicillamine, auranofin or parenteral or oral gold. For inflammatory bowel disease and irritable bowel disorder further convenient agents include sulphasalazine and 5-ASAs, topical and systemic steroids, immunomodulators and immunosuppressants, antibiotics, probiotics and anti-integrins.

The present invention still further relates to the combination of a compound of the invention together with a leukotriene biosynthesis inhibitor, 5-lipoxygenase (5-LO) inhibitor or 5-lipoxygenase activating protein (FLAP) antagonist such as zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2-cyanonaphthalene compounds such as L-739,010; 2-cyanoquinoline compounds such as L-746,530; indole and quinoline compounds such as MK-591, MK-886, and BAY x 1005.

The present invention still further relates to the combination of a compound of the invention together with a receptor antagonist for leukotrienes LTB.sub4., LTC.sub4., LTD.sub4., and LTE.sub4. selected from the group consisting of the phenothiazin-3-ones such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a PDE4 inhibitor including inhibitors of the isoform PDE4D.

The present invention still further relates to the combination of a compound of the invention together with a antihistaminic H.sub1. receptor antagonists such as cetirizine, loratadine, desloratadine, fexofenadine, astemizole, azelastine, and chlorpheniramine.

The present invention still further relates to the combination of a compound of the invention together with a gastroprotective H.sub2. receptor antagonist.

The present invention still further relates to the combination of a compound of the invention together with an α.sub1.- and α.sub2.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents such as ipratropium bromide; tiotropium bromide; oxitropium bromide; pirenzepine; and telenzepine.

The present invention still further relates to the combination of a compound of the invention together with a β.sub1.- to β.sub4.-adrenoceptor agonists such as metaproterenol, isoproterenol, isoprenaline, albuterol, salbutamol, formoterol, salmeterol, terbutaline, orciprenaline, bitolterol mesylate, and pirbuterol; or methylxanthanines including theophylline and aminophylline; sodium cromoglycate; or muscarinic receptor-(M1, M2, and M3) antagonist.

The present invention still further relates to the combination of a compound of the invention together with an insulin-like growth factor type I (IGF-1) mimetic.

The present invention still further relates to the combination of a compound of the invention together with an inhaled glucocorticoid with reduced systemic side effects, such as prednisone, prednisolone, flunisolide, triamcinolone acetonide, beclomethasone dipropionate, budesonide, fluticasone propionate, and mometasone furoate.

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-12.

The present invention still further relates to the combination of a compound of the invention together with other modulators of chemokine receptor function such as CCR1, CCR2, CCR2A, CCR2B, CCR3, CCR4, CCR5, CCR6, CCR7, CCR8, CCR9, CCR10 and CCR11 (for the C-C family); CXCR1, CXCR3, CXCR4 and CXCR5 (for the C-X-C family) and CX₃CR1 for the C-X₃-C family.

The present invention still further relates to the combination of a compound of the invention together with antiviral agents such as Viracept, AZT, aciclovir and famciclovir, and antisepsis compounds such as Valant.

The present invention still further relates to the combination of a compound of the invention together with cardiovascular agents such as calcium channel blockers, lipid lowering agents such as statins, fibrates, beta-blockers, Ace inhibitors, Angiotensin-2 receptor antagonists and platelet aggregation inhibitors.

The present invention still further relates to the combination of a compound of the invention together with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, COX-2 inhibitors, propentofylline or metrifonate.

The present invention still further relates to the combination of a compound of the invention together with (i) tryptase inhibitors; (ii) platelet activating factor (PAP) antagonists; (iii) interleukin converting enzyme (ICE) inhibitors; (iv) IMPDH inhibitors; (v) adhesion molecule inhibitors including VLA-4 antagonists; (vi) cathepsins; (vii) MAP kinase inhibitors; (viii) glucose-6 phosphate dehydrogenase inhibitors; (ix) kinin-B.sub1.- and B.sub2.-receptor antagonists; (x) anti-gout agents, e.g., colchicine; (xi) xanthine oxidase inhibitors, e.g., allopurinol; (xii) uricosuric agents, e.g., probenecid, sulfinpyrazone, and benzbromarone; (xiii) growth hormone secretagogues; (xiv) transforming growth factor (TGFβ); (xv) platelet-derived growth factor (PDGF); (xvi) fibroblast growth factor, e.g., basic fibroblast growth factor (bFGF); (xvii) granulocyte macrophage colony stimulating factor (GM-CSF); (xviii) capsaicin cream; (xix) Tachykinin NK.sub1. and NK.sub3. receptor antagonists selected from the group consisting of NKP-608C; SB-233412 (talnetant); and D-4418; (xx) elastase inhibitors selected from the group consisting of UT-77 and ZD-0892; (xxi) TNFδ converting enzyme inhibitors (TACE); (xxii) induced nitric oxide synthase inhibitors (iNOS) or (xxiii) chemoattractant receptor-homologous molecule expressed on TH2 cells, (CRTH2 antagonists).

The compounds of the present invention may also be used in combination with osteoporosis agents such as roloxifene, droloxifene, lasofoxifene or fosomax and immunosuppressant agents such as FK-506, rapamycin, cyclosporine, azathioprine, and methotrexate.

The compounds of the invention may also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc and P2X7 receptor antagonists.

The compounds of the invention can also be used in combination with existing therapeutic agents for the treatment of cancer. Suitable agents to be used in combination include:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

### Pharmacological Data

### Ligand Binding Assay

[¹²⁵I]IL-8 (human, recombinant) was purchased from Amersham, U.K. with a specific activity of 2,000 Ci/mmol. All other chemicals were of analytical grade. High levels of hrCXCR2 were expressed in HEK 293 cells (human embryo kidney 293 cells ECACC No. 85120602) (Lee et al. (1992) J. Biol. Chem. 267 pp 16283-16291). hrCXCR2 cDNA was amplified and cloned from human neutrophil mRNA. The DNA was cloned into PCRScript (Stratagene) and clones were identified using DNA. The coding sequence was sub-cloned into the eukaryotic expression vector RcCMV (Invitrogen). Plasmid DNA was prepared using Quiagen Megaprep 2500 and transfected into HEK 293 cells using Lipofectamine reagent (Gibco BRL). Cells of the highest expressing clone were harvested in phosphate-buffered saline containing 0.2% (w/v) ethylenediaminetetraacetic acid (BDTA) and centrifuged (200 g, 5 min.). The cell pellet was resuspended in ice cold homogenisation buffer [10 mM HEPES (pH 7.4), 1 mM dithiothreitol, 1 mM EDTA and a panel of protease inhibitors (1 mM phenyl methyl sulphonyl fluoride, 2 μg/ml soybean trypsin inhibitor, 3 mM benzamidine, 0.5 μg/ml leupeptin and 100 μg/ml bacitracin)] and the cells left to swell for 10 minutes. The cell preparation was disrupted using a hand held glass mortar/PTFE pestle homogeniser and cell membranes harvested by centrifugation (45 minutes, 100,000 g, 4° C.). The membrane preparation was stored at −70° C. in homogenisation buffer supplemented with Tyrode's salt solution (137 mM NaCl, 2.7 ml KCl, 0.4 mM NaH₂PO₄), 0.1% (w/v) gelatin and 10% (v/v) glycerol.

All assays were performed in a 96-well MultiScreen 0.45 μm filtration plates (Millipore, U.K.). Each assay contained ˜50 pM [¹²⁵I]IL-8 and membranes (equivalent to ˜200,000 cells) in assay buffer [Tyrode's salt solution supplemented with 10 mM HEPES (pH 7.4), 1.8 mM CaCl₂, 1 mM MgCl₂, 0.125 mg/ml bacitracin and 0.1% (w/v) gelatin]. In addition, a compound of formula (I) according to the Examples was pre-dissolved in DMSO and added to reach a final concentration of 1% (v/v) DMSO. The assay was initiated with the addition of membranes and after 1.5 hours at room temperature the membranes were harvested by filtration using a Millipore MultiScreen vacuum manifold and washed twice with assay buffer (without bacitracin). The backing plate was removed from the MultiScreen plate assembly, the filters dried at room temperature, punched out and then counted on a Cobra gamma-counter.

The compounds of formula (I) according to the Examples 1-7 were found to have pIC₅₀ values of greater than (>) 5.0.

### Intracellular Calcium Mobilisation Assay

Human neutrophils were prepared from EDTA-treated peripheral blood, as previously described (Baly et al. (1997) Methods in Enzymology 287 pp 70-72), in storage buffer [Tyrode's salt solution (137 mM NaCl, 2.7 mM KCl, 0.4 mM NaH₂PO₄) supplemented with 5.7 mM glucose and 10 mM HEPES (pH 7.4)].

The chemokine GROδ (human, recombinant) was purchased from R&D Systems (Abingdon, U.K.). All other chemicals were of analytical grade. Changes in intracellular free calcium were measured fluorometrically by loading neutrophils with the calcium sensitive fluorescent dye, fluo-3, as described previously (Merritt et al. (1990) Biochem. J. 269, pp 513-519). Cells were loaded for 1 hour at 37° C. in loading buffer (storage buffer with 0.1% (w/v) gelatin) containing 5 μM fluo-3 AM ester, washed with loading buffer and then resuspended in Tyrode's salt solution supplemented with 5.7 mM glucose, 0.1% (w/v) bovine serum albumin (BSA), 1.8 mM CaCl₂ and 1 mM MgCl₂. The cells were pipetted into black walled, clear bottom, 96 well micro plates (Costar, Boston, U.S.A.) and centrifuged (200 g, 5 minutes, room temperature).

A compound of formula (I) according to the Examples was pre-dissolved in DMSO and added to a final concentration of 0.1% (v/v) DMSO. Assays were initiated by the addition of an A₅₀ concentration of GROα and the transient increase in fluo-3 fluorescence (δ_{Ex}=490 nm and δ_{Em}=520 nm) monitored using a FLIPR (Fluorometric Imaging Plate Reader, Molecular Devices, Sunnyvale, U.S.A.).

The compounds of formula (1) according to the Examples were tested and found to be antagonists of the CXCR2 receptor in human neutrophils.

The invention will now be illustrated by the following non-limiting Examples in which, unless stated otherwise:
(i) when given Nuclear Magnetic Resonance (NMR) spectra were measured on a Varian Unity Inova 300 or 400 MHz spectrometer. ¹H NMR data is quoted in the form of delta values for major diagnostic protons, given in parts per million (ppm) relative to tetramethylsilane (TMS) as an internal standard.
(ii) Mass Spectrometry (MS) spectra were measured on a Finnigan Mat SSQ7000 or Micromass Platform spectrometer.
(iii) the title and sub-titled compounds of the Examples and methods were named using the ACD/Name program (version 4.55) from Advanced Chemical Development Inc, Canada.
(iv) Normal phase column chromatography and normal phase HPLC was conducted using a silica column. Reverse phase High Pressure Liquid Chromatography (HPLC) purification was performed using either a Waters Micromass LCZ with a Waters 600 pump controller, Waters 2487 detector and Gilson FC024 fraction collector or a Waters Delta Prep 4000 or a Gilson Auto Purification System, using a Symmetry, NovaPak or Ex-Terra reverse phase silica column.
(v) The following abbreviations are used:

AcOH acetic acid

CHCl₃ chloroform

DCM dichloromethane

DMF N,N-dimethylformamide

DMSO dimethylsulfoxide

Et₂O diethyl ether

EtOAc ethyl acetate

MgSO₄ magnesium sulfate

NMP 1-methylpyrrolidin-2-one

THF tetrahydrofuran

H₂O water

### EXAMPLE 1

### N-[4-[[(2,3-difluorophenyl)methyl]thiol]-6-[[(1R)-2-hydroxy-1-methylethyl]amino]-1,3,5-triazin-2-yl]-methanesulfonamide

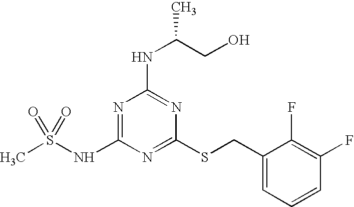

A mixture of methane sulphonamide (0.17 g), tris(dibenzylideneacetone)dipalladium (0) (13 mg), (9,9-dimethyl-9H-xanthene-4,5-diyl)bis[diphenyl-phosphine (8 mg) and cesium carbonate were stirred under a nitrogen atmosphere for 5 minutes. A solution of 2-[[4-chloro-6-[[(2,3-difluorophenyl)methyl]thio]-1,3,5-triazin-2-yl]amino]-(2R)-1-propanol (0.5 g) in anhydrous 1,4-dioxane (5 ml) was added to the above mixture and the reaction was heated to reflux in a pre-heated heat on block for 25 minutes. The reaction mixture was allowed to cool to ambient temperature, diluted with 1N aqueous hydrochloric acid solution and extracted with ethyl acetate (×3). The combined organic layers were dried with magnesium sulfate, filtered and evaporated. The residue was purified by column chromatography on silica using a 99:1 to 98:2 mixture of methylene chloride and methanol as eluent. The resulting solid was further purified by reverse phase HPLC using a 95:5 to 5:95 mixture of 0.2% aqueous ammonium acetate solution and acetonitrile as eluent to give the title compound as a white solid (0.3 g).

NMR Spectrum (CD₃OD) δ 1.21 (m, 3H), 3.33 (m, 3H), 3.58 (m, 2H), 4.19 (m, 1H), 4.56 (m, 2H), 7.16 (m, 2H), 7.38 (m, 1H);

Mass Spectrum: M+H⁺ 406;

Elemental Analysis: Found C, 40.05; H, 4.80; N, 16.48; C₁₄H₁₇F₂N₅O₃S₂.1H₂O requires C, 39.71; H, 4.52; N, 16.54%.

The 2-[[4-chloro-6-[[(2,3-difluorophenyl)methyl]thio]-1,3,5-triazin-2-yl]amino]-(2R)-1-propanol used as a starting material was prepared as follows:—

### i) (2,3-difluorophenyl)methanethiol

Thiourea (6.7 g) was added to a stirred solution of 2,3-difluorobenzylbromide (18.3 g), in ethanol (300 ml). The reaction mixture was heated at reflux for 2.5 hours and then evaporated, treated with 2N sodium hydroxide solution (440 ml) and heated at reflux for a further 4 hours and left stirring at ambient temperature overnight. The resulting mixture was ice-cooled, acidified to pH 6 using concentrated aqueous hydrochloric acid solution and then extracted with diethyl ether. The organic layer was separated, washed with water, dried over anhydrous magnesium sulphate, filtered and evaporated to give (2,3-difluorophenyl)methanethiol (8.0 g). NMR Spectrum: (CDCl₃) δ 1.90 (t, 1H), 3.78 (d, 2H), 7.06 (m, 3H).

### ii) 2-[[4-chloro-6-[[(2,3-difluorophenyl)methyl]thiol]-1,3,5-triazin-2-yl]amino]-(2R)-1-propanol

To an ice-bath cooled solution of cyanuric chloride (3.0 g) and diisopropylethlamine (3.1 ml) in anhydrous tetrahydrofuran (100 ml) was added a solution of (2,3-difluorophenyl)methanethiol (2.6 g) dropwise over 30 minutes. The reaction mixture was stirred at 0° C. under a nitrogen atmosphere for 40 minutes. Further diisopropylethlamine (3.1 ml) was added, followed by a solution of R-(D)-alaninol (1.2 g) in anhydrous tetrahydrofuran (20 ml) dropwise over 5 minutes. The resulting reaction mixture was stirred at 0° C. under a nitrogen atmosphere for 55 minutes. The mixture was diluted with brine and extracted with ethyl acetate (×2). The combined organic layers were dried with magnesium sulfate, filtered and evaporated. The residue was purified by column chromatography on silica using a 90:10 to 70:30 to 60:40 mixture of iso-hexane and ethylacetate as eluent to give 2-[[4-chloro-6-[[(2,3-difluorophenyl)methyl]thio]-1,3,5-triazin-2-yl]amino]-(2R)-1-propanol as a white solid (5.1 g).

NMR Spectrum (DMSO) δ 1.16 (m, 3H), 3.37 (m, 2H), 4.02 (m, 1H), 4.43 (m, 2H), 4.76 (m, 1H), 7.16 (m, 1H), 7.38 (m, 2H), 8.49 (t, 1H);

Mass Spectrum: M+H⁺347/349

### EXAMPLE 2

### N-[4-[[(2,3-difluorophenyl)methyl]thio]-6-[[(1R)-2-hydroxy-1-methylethyl]amino]-1,3,5-triazin-2-yl]-1-azetidinesulfonamide

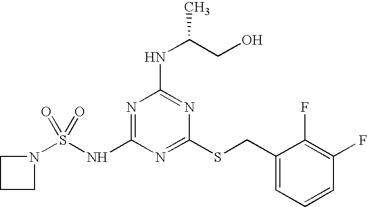

Prepared using the same procedure as for the title compound in example 1; from 2-[[4-chloro-6-[[(2,3-difluorophenyl)methyl]thio]-1,3,5-triazin-2-yl]amino]-(2R)-1-propanol (0.5 g) and azetadine-1-sulfonamide (0.25 g) to give the title compound as a white solid (120 mg).

NMR Spectrum: (CD₃OD) δ 1.18 (m, 3H), 2.17 (quintet, 2H), 3.55 (m, 2H), 4.06 (t, 4H), 4.16 (m, 1H), 4.43 (m, 2H), 7.12 (m, 2H), 7.37 (m, 1H);

Mass Spectrum: M+H⁺447;

Elemental Analysis: Found C, 42.52; H, 4.86; N, 17.68; C₁₆H₂₀F₂N₆O₃S₂.0.3H₂O. 0.3AcOH requires C, 42.43; H, 4.68; N, 17.88%.

### EXAMPLE 3

### N-[4-[[(2,3-difluorophenyl)methyl]thio]-6-[[(1R)-2-hydroxy-1-methylethyl]amino]-1,3,5-triazin-2-yl]-methanesulfonamide

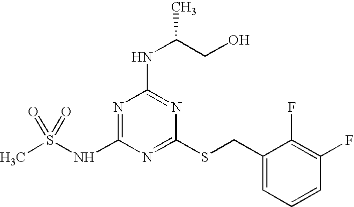

A mixture of methane sulphonamide (0.17 g), tris(dibenzylideneacetone)dipalladium (0) (13 mg), (9,9-dimethyl-9H-xanthene-4,5-diyl)bis[diphenyl-phosphine (8 mg) and cesium carbonate were stirred under a nitrogen atmosphere for 5 minutes. A solution of 2-[[4-chloro-6-[[(2,3-difluorophenyl)methyl]thio]-1,3,5-triazin-2-yl]amino]-(2R)-1-propanol (0.5 g) in anhydrous 1,4-dioxane (5 ml) was added to the above mixture and the reaction was heated to reflux in a pre-heated heat on block for 25 minutes. The reaction mixture was allowed to cool to ambient temperature, diluted with 1N aqueous hydrochloric acid solution and extracted with ethyl acetate (×3). The combined organic layers were dried with magnesium sulfate, filtered and evaporated. The residue was purified by column chromatography on silica using a 99:1 to 98:2 mixture of methylene chloride and methanol as eluent. The resulting solid was further purified by reverse phase HPLC using a 95:5 to 5:95 mixture of 0.2% aqueous ammonium acetate solution and acetonitrile as eluent to give the title compound as a white solid (0.3 g).

Mass Spectrum: [M+H⁺] 406

NMR Spectrum: (CD₃OD) δ: 1.21 (m, 3H), 3.33 (m, 3H), 3.58 (m, 2H), 4.19 (m, 1H), 4.56 (m, 2H), 7.16 (m, 2H), 7.38 (m, 1H);

The 2-[[4-chloro-6-[[(2,3-difluorophenyl)methyl]thio]-1,3,5-triazin-2-yl]amino]-(2R)-1-propanol used as a starting material was prepared as follows:—

### i) (2,3-difluorophenyl)methanethiol

Thiourea (6.7 g) was added to a stirred solution of 2,3-difluorobenzylbromide (18.3 g), in ethanol (300 ml). The reaction mixture was heated at reflux for 2.5 hours and then evaporated, treated with 2N sodium hydroxide solution (440 ml) and heated at reflux for a further 4 hours and left stirring at ambient temperature overnight. The resulting mixture was ice-cooled, acidified to pH 6 using concentrated aqueous hydrochloric acid solution and then extracted with diethyl ether. The organic layer was separated, washed with water, dried over anhydrous magnesium sulphate, filtered and evaporated to give the subtitle compound as an oil. (8.0 g).

NMR Spectrum: (CDCl₃) δ: 1.90 (t, 1H), 3.78 (d, 2H), 7.06 (m, 3H).

### ii) (2R)—2-[[4-chloro-6-[[(2,3-difluorophenyl)methyl]thio]-1,3,5-triazin-2-yl]amino]-1-propanol

To an ice-bath cooled solution of cyanuric chloride (3.0 g) and N,N-diisopropylethlamine (3.1 ml) in anhydrous tetrahydrofuran (100 ml) was added a solution of the product of step (i) (2.6 g) dropwise over 30 minutes. The reaction mixture was stirred at 0° C. under a nitrogen atmosphere for 40 minutes. Further N,N-diisopropylethlamine (3.1 ml) was added, followed by a solution of R-(D)-alaninol (1.2 g) in anhydrous tetrahydrofuran (20 ml) dropwise over 5 minutes. The resulting reaction mixture was stirred at 0° C. under a nitrogen atmosphere for 55 minutes. The mixture was diluted with brine and extracted with ethyl acetate twice. The combined organic layers were dried with magnesium sulfate, filtered and evaporated. The residue was purified by column chromatography on silica using a 90:10 to 70:30 to 60:40 mixture of iso-hexane and ethylacetate as eluent to give subtitle compound as a white solid (5.1 g).

Mass Spectrum: [M+H⁺] 347/349

NMR Spectrum: (DMSO) δ: 1.16 (m, 3H), 3.37 (m, 2H), 4.02 (m, 1H), 4.43 (m, 2H), 4.76 (m, 1H), 7.16 (m, 1H), 7.38 (m, 2H), 8.49 (t, 1H);

### EXAMPLE 4

### N-[4-[[(2,3-difluorophenyl)methyl]thio]-6-[[(1R)-2-hydroxy-1-methylethyl]amino]-1,3,5-triazin-2-yl]-1-azetidinesulfonamide

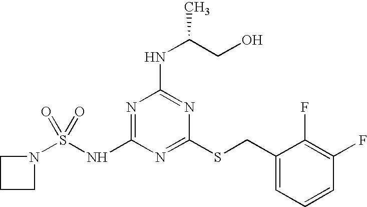

Prepared using the same procedure as for the title compound in example 1; from 2-[[4-chloro-6-[[(2,3-difluorophenyl)methyl]thio]-1,3,5-triazin-2-yl]amino]-(2R)-1-propanol (0.5 g) and azetadine-1-sulfonamide (0.25 g) to give the title compound as a white solid (120 mg).

Mass Spectrum: [M+H⁺] 447

NMR Spectrum: (CD₃OD) δ: 1.18 (m, 3H), 2.17 (quintet, 2H), 3.55 (m, 2H), 4.06 (t, 4H), 4.16 (m, 1H), 4.43 (m, 2H), 7.12 (m, 2H), 7.37 (m, 1H);

### EXAMPLE 5

### 4-morpholinesulfonamide, N-[4-[[(2,3-difluorophenyl)methyl]thio]-6-[[(1R)-2-hydroxy-1-methylethyl]amino]-1,3,5-triazin-2-yl]—

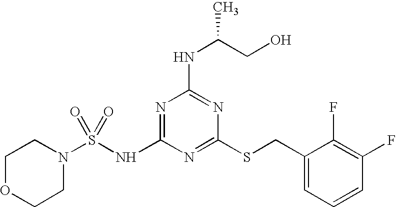

Prepared using the same procedure as for the title compound in example 1; from 2-[[4-chloro-6-[[(2,3-difluorophenyl)methyl]thio]-1,3,5-triazin-2-yl]amino]-(2R)-1-propanol (0.5 g) and morpholine-1-sulfonamide (0.25 g) to give the title compound as a white solid (290 mg).

Mass Spectrum: [M+H⁺] 477

NMR Spectrum: (DMSO) δ: 1.09 (m, 3H), 3.24 (m, 4H), 3.37 (m, 2H), 3.58 (m, 4H), 4.03 (m, 1H), 4.41 (m, 2H), 4.75 (bs, 1H), 7.16 (m, 1H), 7.32 (m, 2H), 7.40-7.72 (m, 2H)

### EXAMPLE 6

### Methanesulfonamide, N-[4-[[2-(2,3-difluorophenoxy)ethyl]thio]-6-[[(1R)-2-hydroxy-1-methylethyl]amino]-1,3,5-triazin-2-yl]—

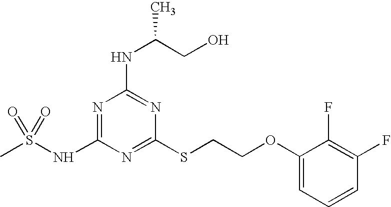

Prepared using the same procedure as for the title compound in example 1; from (2R)-2-[[4-chloro-6-[[2-(2,3-difluorophenoxy)ethyl]thio]-1,3,5-triazin-2-yl]amino]-1-propanol, (0.45 g) and methanesulfonamide (0.14 g) to give the title compound after purification by recrystallisation from ethyl acetate as a white solid (250 mg).

Mass Spectrum: [M+H⁺] 436

NMR Spectrum: (CD₃OD) δ: 1.21 (dd, 3H), 3.36 (m, 3H), 3.54 (m, 4H), 4.16 (m, 1H), 4.34 (m, 2H), 6.82 (q, 1H), 6.94 (t, 1H), 7.07 (m, 1H)

The (2R)-2-[[4-chloro-6-[[2-(2,3-difluorophenoxy)ethyl]thio]-1,3,5-triazin-2-yl]amino]-1-propanol used as a starting material was prepared as follows:—

### (i) (2R)-2-[[4-chloro-6-[[2-(2,3-difluorophenoxy)ethyl]thio]-1,3,5-triazin-2-yl]amino]-1-propanol

Prepared using the same procedure as for the title compound in example 1 step (ii) from cyanuric chloride (0.66 g), 2-(2,3-difluorophenoxy)ethanethiol (0.68 g), and R-(D)-alaninol (270 mg) to give the title compound as a clear oil (0.9 g).

Mass Spectrum: [M+H⁺] 377/379

NMR Spectrum: (DMSO) δ: 1.07 (dd, 3H), 3.37 (m, 2H), 3.46 (m, 2H), 3.97 (m, 1H), 4.35 (q, 2H), 4.76 (m 1H), 7.02 (m, 1H), 7.14 (m 2H), 8.44 (dd, 1H)

### EXAMPLE 7

### Methanesulfonamide, 1,1,1-trifluoro-N-[4-[[(1R)-2-hydroxy-1-methylethyl]amino]-6-(2-phenylethyl)-1,3,5-triazin-2-yl]—

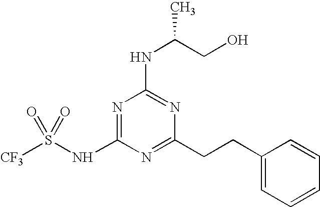

Prepared using the same procedure as for the title compound in example 1; from (2R)-2-[[4-chloro-6-(2-phenylethyl)-1,3,5-triazin-2-yl]amino]-1-propanol (0.29 g) and trifluoromethanesulfonamide (0.22 g) to give the title compound after purification by reverse phase HPLC as a white solid (5 mg).

Mass Spectrum: [M+H⁺] 406

NMR Spectrum: (CD₃OD) δ: 1.23 (d, 3H), 2.81 (t, 2H), 3.03 (t, 2H), 3.65 (dd, 1H), 3.63 (dd, 1H), 4.20-4.27 (m, 1H), 7.16-7.29 (m, 5H)

The (2R)-2-[[4-chloro-6 (2-phenylethyl)-1,3,5-triazin-2-yl]amino]-1-propanol used as a starting material was prepared as follows —

### (i) (2R)-2-[[4-chloro-6-(2-phenylethyl)-1,3,5-triazin-2-yl]amino]-1-propanol

A solution of phenethyl bromide (1.82 ml) in dry diethyl ether (12 ml) was treated with magnesium turnings (0.3 g) under nitrogen atmosphere with gentle warming until all the magnesium had reacted to produce a solution of the Grignard reagent. This solution was then added dropwise to a solution of cyanuric chloride (1.84 g) in benzene (10 ml) at 0° C. with stirring. After stirring at this temperature for 2 h the mixture was allowed to warm to ambient temperature for a further 16 h. The mixture was then treated with N,N-diisopropylethylamine (5.3 ml) followed by R-(D)-alaninol (2.3 ml) and the whole allowed to stir for a further 48 h The mixture was concentrated under vacuo and the residue partitioned between dichloromethane and water. The organic layer was collected and further washed with 2M hydrochloric acid, saturated sodium bicarbonate solution and water again. The organic layer collected, dried (MgSO₄) and solvent evaporated to leave the subtitle product as an orange oil (3 g). Mass Spectrum: [M+H⁺] 293.

### REFERENCE MARKS IN THE DRAWINGS

1 Magnet
2 Yoke
3 Upper plate
4 Magnetic circuit
5 Magnetic gap
6 Frame
7 Diaphragm
8 Voice coil
10 Terminal
10A Center section
10B Movable end
10C and 10D Stopper
11A and 11B Loudspeaker
21 Magnet
22 Yoke
23 Upper plate
24 Magnetic circuit
25 Magnetic gap
26 Frame
27 Diaphragm
28 Voice coil
30 Terminal
30A Stopper
30B Reinforced section
30C Reinforcing rib
30D Center section
30E Contact point
30F Main section
35 Loudspeaker
40 Electronic circuit
60 Display module
70 Outer package
80 Portable telephone unit

1. A compound of formula (1), or a pharmaceutically acceptable salt, solvate or in vivo hydrolysable ester thereof: 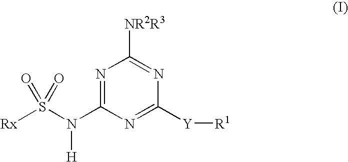
wherein
Y is selected from a bond, —S—, —O—, —NR⁵—, —CF₂—CH₂—, —CF₂CF₂—, —CONR⁵—, phenyl or heteroaryl;
R¹ is a group selected from C₃₋₇carbocyclyl, C₁₋₈alkyl, C₂₋₆alkenyl and C₂₋₆alkynyl, which group is optionally substituted by 1, 2 or 3 substituents independently selected from fluoro, nitrile, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, phenyl or heteroaryl, and wherein phenyl and heteroaryl are optionally substituted by 1, 2 or 3 substituents independently selected from halo, cyano, nitro, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, C₁₋₆alkyl and trifluoromethyl;
R² is C₃₋₇carbocyclyl, optionally substituted by 1, 2 or 3 substituents independently selected from fluoro, —OR⁴, —NR⁵R⁶—CONR⁵R⁶, —COOR⁷, —NR⁸COR⁹, —SR¹⁰, —SO₂R¹⁰, —SO₂NR⁵R⁶, —NR⁸SO₂R⁹;
or R² is a 3-8 membered ring optionally containing 1, 2 or 3 atoms selected from O, S, —NR⁸ and which ring is optionally substituted by C₁₋₃alkyl or fluoro;
or R² is a phenyl or heteroaryl, each of which is optionally substituted by 1, 2 or 3 substituents independently selected from halo, cyano, nitro, —OR⁴, —NR⁵R⁶, —CONR⁵R⁶, —NR⁸COR⁹, —SO₂NR⁵R⁶, —NR⁸SO₂R⁹, C₁₋₆alkyl and trifluoromethyl;
or R² is a group selected from C₁₋₈alkyl, C₂₋₆alkenyl or C₂₋₆alkynyl, which group is substituted by 1, 2 or 3 substituents independently selected from hydroxy, amino, C₁₋₆alkoxy, C₁₋₆alkylamino, di(C₁₋₆alkyl)amino, N—(C₁₋₆alkyl)-N-(phenyl)amino, N—C₁₋₆alkylcarbamoyl, N,N-di(C₁₋₆alkyl)carbamoyl, N—(C₁₋₆alkyl)-N-(phenyl)carbamoyl, carboxy, phenoxycarbonyl, —NR⁸COR⁹, —SO₂R¹⁰, —SO₂NR⁵R⁶ and —NR⁸SO₂R⁹;
R³ is hydrogen or independently R²;
R⁴ is hydrogen or a group selected from C₁₋₆alkyl and phenyl, which group is optionally substituted by 1 or 2 substituents independently selected from halo, phenyl, —OR¹¹ and —NR¹²R¹³;
R⁵ and R⁶ are independently hydrogen or a group selected from C₁₋₆alkyl and phenyl, which group is optionally substituted by 1, 2 or 3 substituents independently selected from halo, phenyl, —OR¹⁴, —NR¹⁶R¹⁶, —COOR¹⁴, —CONR¹⁵R¹⁶, —NR¹⁵COR¹⁶, —SO₂R¹⁰, —SONR¹⁵R¹⁶ and NR¹⁵SO₂R¹⁶;
or R⁵ and R⁶ together with the nitrogen atom to which they are attached form a 4- to 7-membered saturated heterocyclic ring system optionally containing a further heteroatom selected from oxygen and nitrogen atoms, which ring is optionally substituted by 1, 2 or 3 substituents independently selected from phenyl, —OR¹⁴, —COOR¹⁴, —NR¹⁵R¹⁶, —CONR¹⁵R¹⁶, —NR¹⁵COR¹⁶, —SO₂R¹⁰, —SONR¹⁵R¹⁶, NR¹⁵SO₂R¹⁶ or C₁₋₆alkyl (optionally substituted by 1 or 2 substituents independently selected from halo, —NR¹⁵R¹⁶ and —OR¹⁷ groups);
R¹⁰ is hydrogen or a group selected from C₁₋₆alkyl or phenyl, which group is optionally substituted by 1, 2 or 3 substituents independently selected from halo, phenyl, —OR¹⁷ and —NR¹⁵R¹⁶; each of R⁷, R⁸, R⁹, R¹¹, R¹², R¹³, R¹⁴, R¹⁵, R¹⁶, R¹⁷ is independently hydrogen, C₁₋₆alkyl or phenyl;
R^{x} is trifluoromethyl, —NR⁵R⁶, phenyl, napthyl, monocyclic or bicyclic heteroaryl which heteroring may be partially or fully saturated and one or more ring carbon atoms may form a carbonyl group, and wherein each phenyl or heteroaryl group is optionally substituted by 1, 2 or 3 substituents independently selected from halo, cyano, nitro, —OR⁴, —NR⁵R⁶, —CONR⁵R⁶, —COR⁷, —COOR⁷, —NR⁸COR⁹, —SR¹⁰, —SO₂R¹⁰, —SO₂NR⁵R⁶, —NR⁸SO₂R⁹, C₁₋₆alkyl or trifluoromethyl;
or R^{x} is a group selected from C₃₋₇carbocyclyl, C₁₋₈alkyl, C₂₋₆alkenyl and C₂₋₆alkynyl, which group is optionally substituted by 1, 2 or 3 substituents independently selected from halo, —OR⁴, —NR⁵R⁶, —CONR⁵R⁶, —COR⁷, —COOR⁷, —NR⁸COR⁹, —SR¹⁰, SO₂R¹⁰, —SO₂NR⁵R⁶, —NR⁸SO₂R⁹, phenyl or heteroaryl, and wherein each phenyl or heteroaryl group is optionally substituted by 1, 2 or 3 substituents independently selected from halo, cyano, nitro, —OR⁴, —NR⁵R⁶, —CONR⁵R⁶, —COR⁷—COOR⁷, —NR⁸COR⁹, —SR¹⁰, —SO₂R¹⁰, —SO₂NR⁵R⁶, —NR⁸SO₂R⁹, C₁₋₆alkyl or trifluoromethyl.

2. A compound, or a pharmaceutically acceptable salt, solvate or in vivo hydrolysable ester thereof according to claim 1 wherein R² is C₁₋₈alkyl optionally substituted by 1 or 2 hydroxy substituents.

3. A compound, pharmaceutically acceptable salt, solvate or in vivo hydrolysable ester thereof according to claim 1 wherein R¹ is benzyl or —CH₂CH₂OPh, or CH₂CH₂Ph wherein in each case the phenyl ring is optionally substituted by 1, 2 or 3 substituents independently selected from fluoro, chloro, bromo, methoxy, methyl and trifluoromethyl.

4. A compound, pharmaceutically acceptable salt, solvate or in vivo hydrolysable ester thereof according to claim 1 wherein R³ is hydrogen.

5. A compound, pharmaceutically acceptable salt, solvate or in vivo hydrolysable ester thereof according to claim 1 wherein Y is selected from a bond, —S—, and —CF₂—CH₂— and —CH₂—CH₂—.

6. A compound, pharmaceutically acceptable salt, solvate or in vivo hydrolysable ester thereof according to claim 1 wherein R^{x} is methyl, 1-methylimidazolyl, 1,2-dimethylimidazolyl, N,N-dimethylamino, azetidinyl, pyrrolidinyl, morpholinyl, piperidinyl and trifluoroethyl.

7. A compound selected from the group consisting of:
N-[4-[[(2,3-difluorophenyl)methyl]thio]-6-[[(1R)-2-hydroxy-1-methylethyl]amino]-1,3,5-triazin-2-yl]-methanesulfonamide;
N-[4-[[(2,3-difluorophenyl)methyl]thio]-6-[[(1R)-2-hydroxy-1-methylethyl]amino]-1,3,5-triazin-2-yl]-1-azetidinesulfonamide;
N-[4-[[(2,3-difluorophenyl)methyl]thio]-6-[[(1R)-2-hydroxy-1-methylethyl]amino]-1,3,5-triazin-2-yl]-methanesulfonamide;
N-[4-[[(2,3-difluorophenyl)methyl]thio]-6-[[(1R)-2-hydroxy-1-methylethyl]amino]-1,3,5-triazin-2-yl]-1-azetidinesulfonamide;
4-morpholinesulfonamide, N-[4-[[(2,3-difluorophenyl)methyl]thio]-6-[[(1R)-2-hydroxy-1-methylethyl]amino]-1,3,5-triazin-2-yl]-;
methanesulfonamide, N-[4-[[2-(2,3-difluorophenoxy)ethyl]thio]-6-[[(1R)-2-hydroxy-1-methylethyl]amino]-1,3,5-triazin-2-yl]-; and
methanesulfonamide, 1,1,1-trifluoro-N-[4-[[(1R)-2-hydroxy-1-methylethyl]amino]-6-(2-phenylethyl)-1,3,5-triazin-2-yl]-;
or a pharmaceutically acceptable salt, solvate or in vivo hydrolysable ester thereof.

8-13. (canceled)

14. A pharmaceutical composition comprising a compound, or a pharmaceutically acceptable salt, solvate or in vivo hydrolysable ester thereof according to claim 1, and a pharmaceutically-acceptable diluent or carrier.

15. A process for the preparation of a compound according to claim 1 or a pharmaceutically acceptable salt, solvate or in vivo hydrolysable ester thereof, which comprises the steps of:
treating a compound of formula (2): 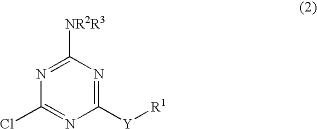
wherein Y, R¹, R² and R³ are as defined in claim 1, with a sulfonamide of formula R^{x}SO₂NH₂ where R^{x} is as defined in claim 1;
and optionally thereafter, one or more of steps (i), (ii), (iii), (iv), or (v) in any order:
i) removing any protecting groups;
ii) converting the compound of formula (1) into a further compound of formula (1);
iii) forming a salt;
iv) forming a prodrug;
v) forming an in vivo hydrolysable ester.

16. A combination therapy which comprises administering a compound of formula (1) or a pharmaceutically acceptable salt, solvate or in vivo hydrolysable ester thereof, or a pharmaceutical composition or formulation comprising a compound of formula (1), concurrently or sequentially with other therapy and/or another pharmaceutical agent.

17. The combination therapy as claimed in claim 16 for the treatment of asthma, allergic rhinitis, COPD, inflammatory bowel disease, irritable bowel syndrome, osteoarthritis, osteoporosis, rheumatoid arthritis, or psoriasis.

18. The combination therapy as claimed in claim 16 for the treatment of cancer.

19. A pharmaceutical composition which comprises a compound of formula (1) according to claim 1 or a pharmaceutically acceptable salt, solvate or in vivo hydrolysable ester thereof, in conjunction with another pharmaceutical agent.

20-21. (canceled)

22. A method of treating a disease or medical condition selected from asthma, allergic rhinitis, COPD, inflammatory bowel disease, osteoarthritis, osteoporosis, rheumatoid arthritis, or psoriasis in a warm-blooded animal in need thereof, the method comprising administering to said animal an effective amount of a compound of claim 1, or a pharmaceutically acceptable salt, solvate or in vivo hydrolysable ester thereof.

23. A method of treating cancer in a warm-blooded animal in need thereof, the method comprising administering to said animal an effective amount of a compound of claim 1, or a pharmaceutically acceptable salt, solvate or in vivo hydrolysable ester thereof.

24. A method of treating a disease or medical condition mediated by the modulation of chemokine receptor activity, the method comprising administering to said animal an effective amount of a compound of claim 1, or a pharmaceutically acceptable salt, solvate or in vivo hydrolysable ester thereof.
